(19) United States
(12) Patent Application Publication
(10) Pub. No.: US 2010/0152602 A1
(43) Pub. Date: Jun. 17, 2010

(54) WHOLE BODY ELECTROMAGNETIC DETECTION SYSTEM

(76) Inventor: Colin A. Ross, McKinney, TX (US)

Correspondence Address: CARR LLP, 670 FOUNDERS SQUARE, 900 JACKSON STREET, DALLAS, TX, 75202, US

(21) Appl. No.: 12/337,451

(22) Filed: Dec. 17, 2008

(51) Int. Cl. A61B 5/04

(52) U.S. Cl. 600/544; 600/546

(57) ABSTRACT

An apparatus and method for characterizing electrical signals from a living organism comprising sensors configured to be positioned to receive the electrical signals emanating from a human signal source. A processor may be configured to interpret readings made by the sensors to output a characterization of the electrical signals.

### BACKGROUND OF THE INVENTION

1. Field of the Invention

The present invention relates to an electromagnetic detection system of the electromagnetic fields emanating from a living organism and, more particularly, to the characterization of electromagnetic fields emanating from the human body.

2. Description of the Related Art

All biological systems generate electromagnetic fields (EMF) and these fields interact with and are affected by the magnetic field surrounding the earth as well as other sources of EMF such as solar flares. The human body in particular generates a relatively complex electromagnetic field. There currently exist known methods of measuring the electromagnetic field of a body. The electromagnetic field generated by the brain, for example, can be measured with a highly sensitive instrument such as a Superconducting Quantum Interference Device (SQUID) magnetometer. However, since the magnetic field generated by the brain is on the order of roughly one billion times weaker than the main magnetic field of the earth, most SQUID magnetometers are typically housed in magnetically insulated rooms in order to eliminate the background noise that would otherwise overwhelm the signal from the brain. Such full-size rooms can cost approximately $250,000 to construct and a SQUID magnetometer capable of taking a full brain map costs about $2 million.

A less costly way to measure the electrical field generated by the brain is through the use of a contacting electroencephalogram (EEG) system. A simple EEG software program and the necessary leads and electrodes can be purchased for about $1,200 and run on a laptop computer. A system such as this is commonly used during biofeedback treatment by psychologists. Biofeedback is the process of monitoring a physiological signal, and amplifying, conditioning, and displaying the signal to the monitored subject so that he or she can observe small changes in the signal. Gradually, through trial and error, the monitored subject may learn to affect certain biological or physiological processes by associating certain actions with the subsequent changes in the monitored signal.

Additionally, in some situations the measurement of electric fields produced by certain portions of the body may be useful in identifying certain medical conditions or in the development of medical treatments. For example, a typical application involves the measurement of the electrical field of the heart through the use of a contacting electrocardiogram (ECG or EKG). The printout of the measurement may be used in making a number of different diagnoses, including the likelihood of a heart attack, and the identification of abnormal electrical conduction within the heart, among others. These methods require that detection of the electrical field be accomplished using a contacting sensor, such as an electrode.

Researchers have developed electrical potential probes, as a type of non-contact electrode that detects the electric potentials of a living organism generated by electrical currents of the body. Harland C. J., “Electrical Potential Probes—New Directions in the Remote Sensing of the Human Body” Meas. Sci. Technol., Vol. 12 2002, pp. 163-169. These electrodes do not require electrical charge contact with the living organism to detect the electromagnetic fields emanating from the body. These researchers have demonstrated that by using ultra-high-impedance electrodes, the electrical field of a heart (ECG) can be detected with the electrode at up to one meter away from the body. The use of these non-contacting electrodes has given medical researchers and practitioners the option to detect the electrical field of living organisms in a non-invasive manner.

### SUMMARY

An apparatus and method for characterizing electrical signals emanating from a living organism are provided, comprising an array of sensors configured to be positioned to receive the electrical signals and deliver readings corresponding to the electrical signals to a processor for interpreting the readings.

### BRIEF DESCRIPTION OF DRAWINGS

For a more complete understanding of the present invention and the advantages thereof, reference is now made to the following Detailed Description taken in conjunction with the accompanying drawings, in which:

FIG. 1 is a schematic of an embodiment of a whole body scanner assembly;

FIG. 2 is a schematic of an embodiment of a system for characterizing the EMF emanating from a human body.

FIG. 3A is a side view of an alternate embodiment of a whole body scanner assembly;

FIGS. 3B-3D are side views of the whole body scanner assembly of FIG. 3A showing a sensor carrier in three different positions;

FIGS. 4A and 4B are a bottom view and a side view, respectively, of one embodiment of a sensor carrier for the whole body scanner assembly shown in FIG. 3A;

FIG. 5 is a perspective view of a hand held embodiment of a whole body scanner assembly;

FIG. 6 is a perspective view of one embodiment of a stationary electromagnetic scanner;

FIGS. 7A and 7B is a first and second bottom view of a sensor housing;

FIG. 8 is a perspective view of a sensor housing having a generally curved shape;

FIG. 9A is a first front view of an alternate embodiment of a whole body scanner assembly comprising a head covering for a human body;

FIG. 9B is a second front view of the embodiment of a whole body scanner assembly shown in FIG. 9A showing the whole body scanner coupled to a head portion of a human body;

FIG. 10 is a cross-sectional view of the whole body scanner assembly shown in FIG. 9A taken along line 10-10, as shown in FIG. 9A;

FIG. 11 is a cross-sectional view of the whole body scanner assembly shown in FIG. 9A taken along line 11-11;

FIG. 12A is a front view of an embodiment of a whole body scanner comprising a garment intended to be worn as a shirt by a human subject;

FIG. 12B is a top view of the embodiment of the whole body scanner shown in FIG. 12A;

FIG. 13A is a cross-sectional view of the garment shown in FIG. 12 taken along line 13-13;

FIG. 13B is a zoomed view of section 13B of FIG. 13A; and

FIG. 14 is a flow diagram of the operations of a method for characterizing the electrical signals emanating from a living organism.

### DETAILED DESCRIPTION

In the following discussion, numerous specific details are set forth to provide a thorough understanding of the present invention. However, those skilled in the art will appreciate that the present invention may be practiced without such specific details. In other instances, well-known elements have been illustrated in schematic or block diagram form in order not to obscure the present invention in unnecessary detail. Additionally, for the most part, details concerning network communications, electro-magnetic signaling techniques, and the like, have been omitted inasmuch as such details are not considered necessary to obtain a complete understanding of the present invention, and are considered to be within the understanding of persons of ordinary skill in the relevant art.

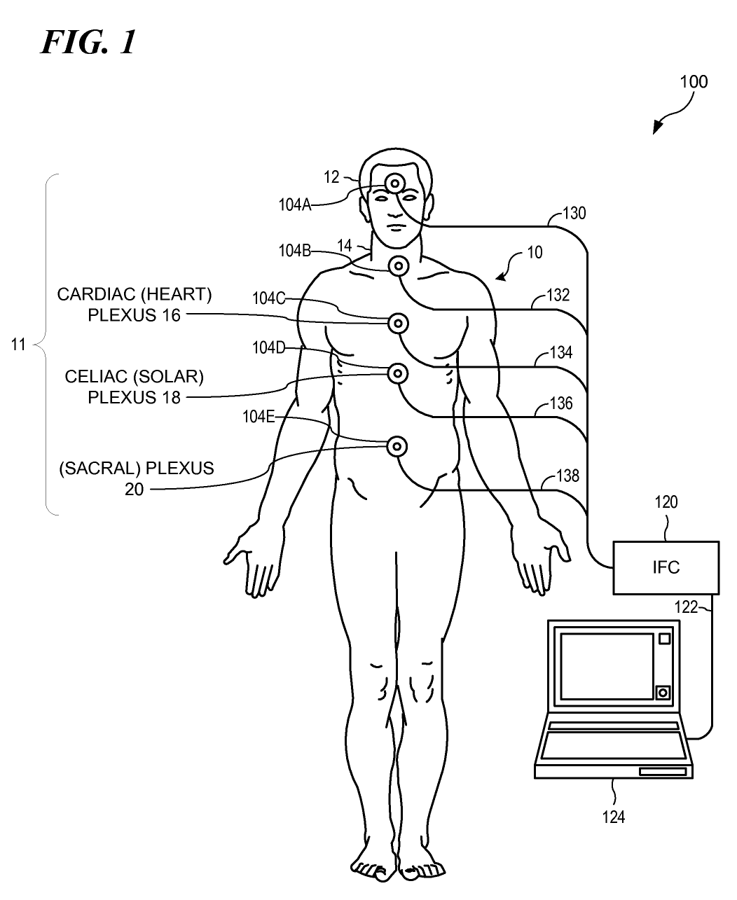

Turning now to FIG. 1, there is shown an illustrative schematic example of an embodiment of a whole body scanner assembly 100 for receiving and detecting a plurality of electrical body signals from a living organism. In the embodiment shown, an 8-lead neurofeedback system 102 comprising a plurality of sensors, such as first electrode 104a, second electrode 104b, third electrode 104c, fourth electrode 104d, fifth electrode 104e, sixth electrode 104f, seventh electrode 104g, and eighth electrode 104h.

The electrodes 104a-104h may be configured to make electrical contact with the surface skin of the human body. Each electrode 104a-104h may be connected individually to one or more target body portions of a human body 10. The target body portions (collectively referred to as reference numeral 11) of the human body 10 may comprise nerve centers of the human body where nerve activity and electromagnetic activity may be relatively high.

The electrodes 104a-104h may be configured for attachment to the human body 10. In some embodiments, the electrodes may comprise voltage probes, such as silver metal electrodes, pasted to the skin using an adhesive. An electrolytic paste, such as silver chloride gel, may interface between the skin and the electrode to detect the flow of electric current in the skin.

In some embodiments, the electrodes 104a-104h may each comprise an input impedance value sufficient to reliably receive electrical signals at a distance D. The electrode 220 may have an input impedance value from about 10⁷Ω up to approximately 10¹⁵Ω. By comparison, conventional paste-on sensors have impedance values approximately in the range of 10⁶ to 10⁷Ω.

It may be advantageous to utilize a high input impedance electrode in the whole body scanner assembly 100. Such electrodes may be used for on-body sensing, even though the electrode remains electrically insulated from the skin. The electrodes may not require a charge contact with a skin surface of the human body, unlike conventional paste-on sensors. The high input impedance electrodes may be taped to the human body with adhesive tape. The electrodes may be used in pairs to obtain a differential signal to eliminate unwanted body noise sources of electrical activity. The noise floors of high impedance electrical potential electrodes may be on the order of approximately 4 ηV Hz^{-1/2} to 70 ηV Hz^{-1/2} at 1 Hz., depending on whether the electrodes are single-ended or coupled for differential readings.

In the embodiment shown, only electrodes 104a-104e are shown connected to the human body 10. It should be understood by persons of ordinary skill in the art that the number of electrodes used and attached may vary. Also, more than one electrode may be attached to a single target body portion 11. Each target body portion 11 may be accessed by a single-ended or a coupled pair of electrons. In the embodiment shown in FIG. 1, single-ended electrodes are utilized.

A first electrode 104a may be attached to a head portion 12, such as a forehead, of the human body 10. A second electrode 104b may be attached to a throat portion 14 of the human body 10. A third electrode 104c may be attached to a chest portion, such as cardiac plexus 16. A fourth electrode 104d may be attached to an upper abdomen portion, such as celiac plexus 18. A fifth electrode 104e may be attached to lower abdomen portion, such as sacral plexus 104e. Each electrode 104a-104e may couple to a first end of each of lead wires 130, 132, 134, 136, and 138.

The whole body scanner assembly 100, as shown in FIG. 1, may further comprise a receiver, such as an interface (IFC) 120, coupled to a second end of each the lead wires 130, 132, 134, 136, and 138. The IFC 120 may comprise a processor configured for receiving data corresponding to readings from the electrodes 104a-104h through each respective lead wire 130, 132, 134, 136, and 138. The processor of the IFC 120 may include a computer readable storage medium configured to embody software instructions for operating the processor. The data received by the IFC 120 may correspond to the electrical signals received from the portion of the body each respective electrode 104a-104h is attached. In some embodiments, the IFC 120 may comprise a wireless receiver for receiving wireless signals transmitted from the electrodes 104a-104h. In a wireless configuration, the lead wires 130, 132, 134, 136, and 138 may not be needed. The IFC 120 may further process and filter the body signals for transmission to a first computer 124.

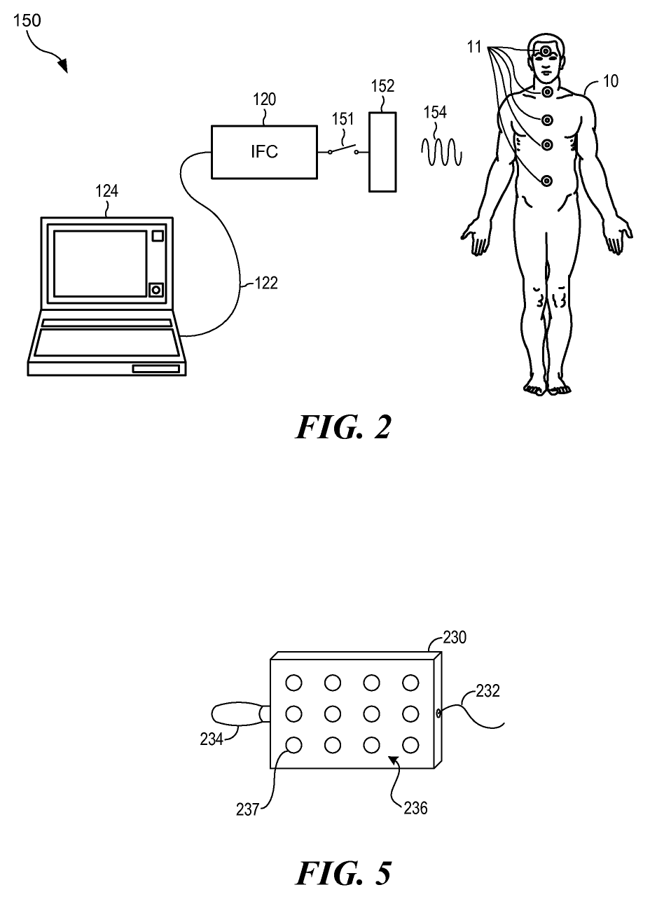

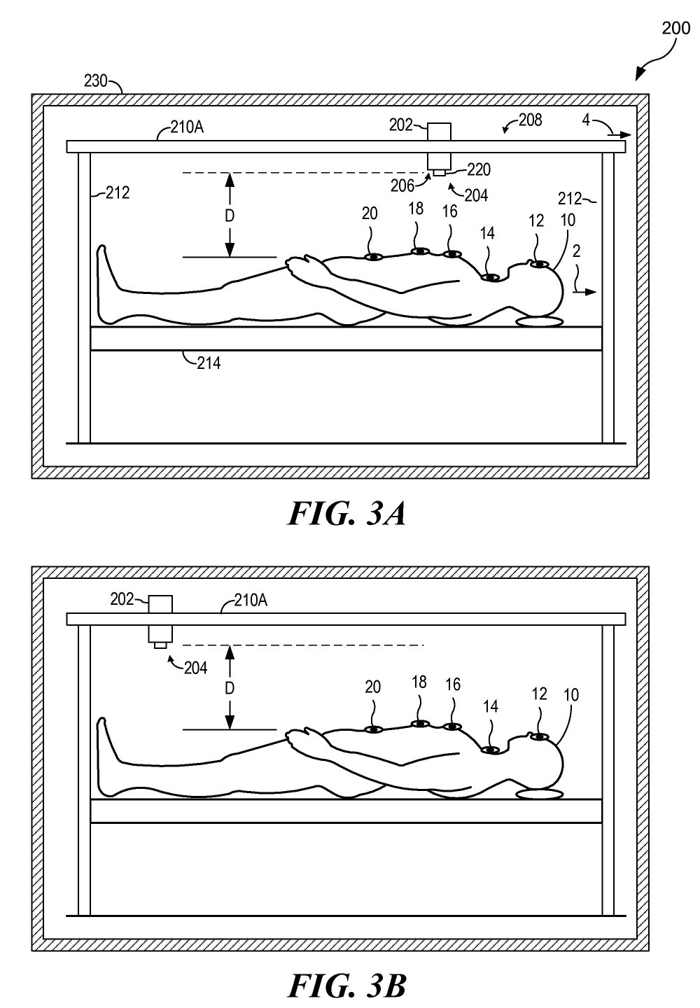

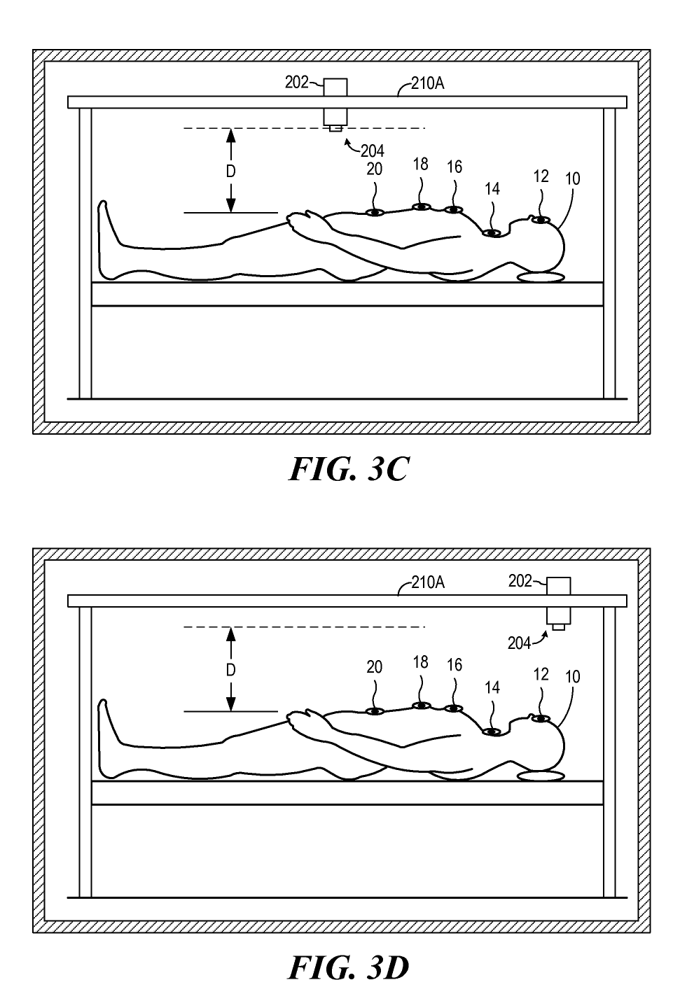

Turning now to FIG. 2, there is shown a schematic diagram of the components of a system for characterizing the electromagnetic field emanating from a living organism such as the human body 10. The system 150 may comprise sensor 152 configured to receive and detect electrical signals 154 from the human body 10, which may comprise a signal source. The electrical signals may originate from target body portions 11, such as those described in FIG. 1. The sensor 152 may be a contacting sensor, such as electrodes 104a-104h as described in FIG. 1 or non-contacting sensors, as described later in FIGS. 3A-8. The system 150 may comprise one or more processors, such as the IFC 120 and first computer 124, where IFC 120 has the functionality described in FIG. 1. The IFC 120 may be configured with a switching mechanism 151 to establish an electrical connection to the sensor 154 for receiving the readings of the electrical signals 154 from the sensor 154. In some embodiments, the switching mechanism may be incorporated into the sensor 152.

Referring now to FIGS. 1 and 2, the first computer 124 may receive processed signals from the IFC 120 via a first connection 122. The first computer 124 may comprise a processor configured for receiving software instructions and a computer readable storage medium having software code for giving instructions to the processor of the first computer. In some embodiments, the computer readable storage medium may comprise software instructions for interpreting the plurality of body signals received from the electrodes 104a-104h. The first computer 124 may take the processed information gathered from the body signals 154 and transform them into waveforms, numerical values corresponding to electrical and magnetic characteristics of the body signals, such as voltage, frequency and amplitude, or into a color-coded scheme, such as a tomographic map of the body signals. The first computer 124 may further output the information to a computer display, a printer, or other device configured receive and handle outputted information, such as a hard drive, a flash drive, a compact disc medium, or other medium for storing the data. The data may be stored in various formats including image, video, or database formats. It should be understood that the functions of the IFC 120 and the first computer 124 may be performed by one or more circuits or one or more processors and may be located local or remote to the whole body scanner 100.

In those embodiments where the output is displayed to a monitor or recorded to a video file, the display may represent a real-time characterization of the body signals 154. The body signals 154 may be displayed as static images, a series of images, an average value with standard deviation over a period of time, or a real-time fluctuating display. The images outputted to the display may comprise one-, two-, three- or multi-dimensional representations of the body signals 154. The display may further be configured to show the effects of environmental inputs, or stimuli, on the human body electromagnetic field.

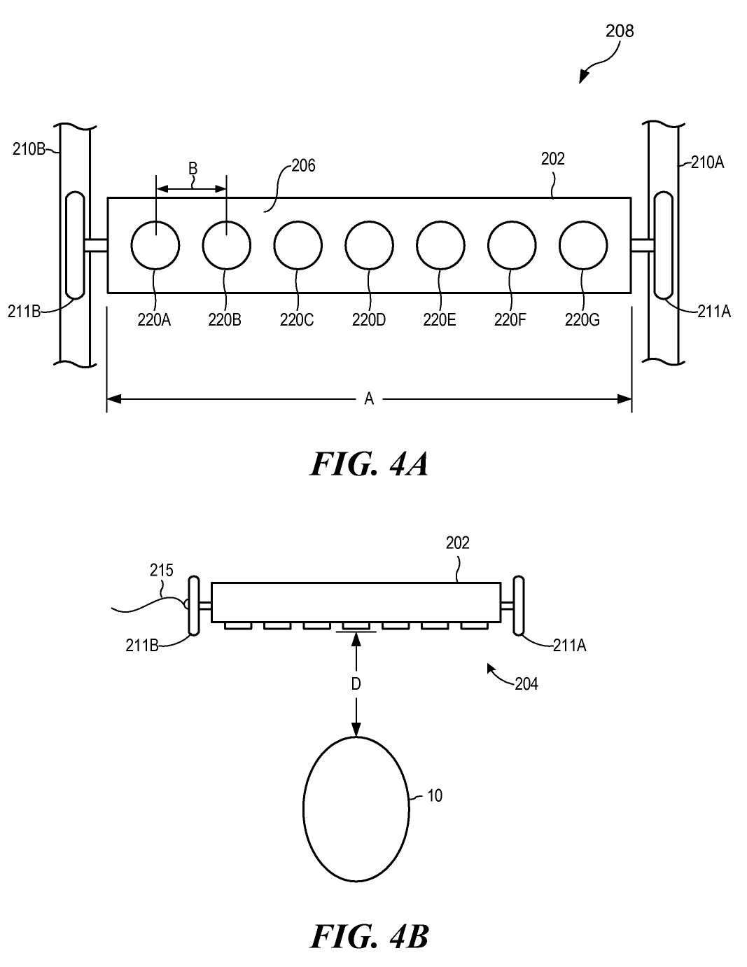

Turning now to FIG. 3A, there is shown another embodiment for a whole body scanner assembly 200 for interpreting received signals from the targeted portions (12, 14, 16, 18, and 20) of the human body 10. The whole body scanner assembly 200 may comprise a sensor carrier 202. An array of sensors 204, comprising a first electrode 220a, a second electrode 220b, a third electrode 220c, a fourth electrode 220d, a fifth electrode 220e, a sixth electrode 220f, and a seventh electrode 220g (shown collectively as reference numeral 220 in FIG. 3A and individually in FIGS. 4A and 4B) may be configured for mounting to a bottom surface 206 of the sensor carrier 204, such that a receiving portion of each of the electrodes 220a-220g may face away from the bottom surface 206. Seven electrodes 220a-220g are shown in FIGS. 5A and 5B; however, it should be understood by a person of ordinary skill in the art that more or less electrodes may be utilized to characterize the electric signals emanating from the human body 10.

The sensor carrier 202 may be mounted to a scanning mechanism 208. The scanning mechanism 208 may be configured for moving the sensor carrier 202 along a scanning path 4, which in some embodiments may be parallel to a body axis 2. The scanning path 4 may correspond to a substantially straight line along which the sensor carrier 202 may travel when in operation. It should be recognized by persons of ordinary skill in the art that the scanning path 4 may comprise curved, zig-zag, or other configurations, which may depend on the target body portions 12, 14, 16, 18, and 20 of the human body 10.

The body axis 2 may correspond to a length of a human body, such as from head to toe. The body axis 2 may further comprise generally an axis of intended target body portions 12, 14, 16, 18, and 20 emanating body signals. In other embodiments, the body axis 2 may be chosen differently to facilitate receiving body signals from a different length of the body.

Turning now to FIGS. 4A and 4B, there are shown a bottom view and a side view, respectively, of one embodiment of the sensor carrier 202. In FIG. 4A, the electrodes 220a-220g are shown set up in the sensor array 204. The sensor array 204 shown is a relatively straight line of electrodes 220a-220g spanning a length A, as shown. Each of the electrodes 220a-220g may be set at a gap B from each respective neighboring electrode.

The sensor array 204 may have other geometric configurations. The length A and the gap B may be varied to achieve an optimum characterization of the electric field emanation from the human body 10. In other embodiments, the sensor array 204 may comprise sensors aligned in staggered or aligned rows. The gap B between sensors may be optimized depending on the target body portions. It should be recognized by persons of ordinary skill in the art that the sensor carrier 202 may be configured to allow for the configuration of the sensor array 204 to be varied to meet individual requirements of the human body 10.

In the embodiment shown in FIGS. 3A and 4A, the scanning mechanism 208 may comprise tracks 210a and 210b for receiving translation members 211a and 211b of the sensor carrier 202. The translation members 211a and 211b may couple into each respective track 210a and 210b. The translation members 211a and 211b may be configured to receive a movement force, such as a torque for moving the sensor carrier 202 along each track. The tracks 210a and 210b may be substantially parallel, as shown in FIG. 4A. The movement force may be applied to the translation members 211a and 211b via a motor (not shown), or other known device such as a pulley for generating a movement force.

As shown in FIG. 4B, the sensor carrier 202 may be mounted at a distance D from a reference point such as the human body 10 (as shown in FIGS. 3 and 4B) or a receiving platform 214 (shown in FIG. 3). Turning to FIG. 3A, the scanning mechanism 208 may comprise a support structure having a plurality of support members 212 for setting the tracks 210a and 210b at the distance D. In the embodiment shown, the tracks 210a and 210b (not shown) have been set substantially level relative to the receiving platform 214, so that when the sensor carrier 202 travels along the scanning path 4 the sensor carrier 202 may stay at substantially the distance D from the target body portions 11. In this configuration, the sensor carrier 202 may move along the tracks 210a and 210b at substantially the same distance D from the human body 10. The support members 212 may be configured to be adjustable to fix the distance D.

Turning now to FIGS. 3B, 3C, and 3D, there are shown side views of the whole body scanner assembly 200 of FIG. 3A with the sensor carrier 202 shown in three positions. In FIG. 3B, the sensor carrier 202 is shown suspended substantially above a lower leg portion of the human body 10. As described in FIGS. 4A and 4B, the sensor carrier 202 may be moved along the tracks 210A and 210B. In the embodiment shown, the sensor carrier 202 may begin at the lower leg portion and travel substantially parallel the body axis 2 (shown in FIG. 3A) and along the scanning path 4 (shown in FIG. 3A) towards the head portion 12. As shown in FIGS. 3C and 3D, the sensor carrier 202 may be actuated along the scanning path 4 and along the body axis 2 to cross over the target body portions 20, 18, 16, 14, and 12. The sensor array 204 may take readings corresponding to the electrical signals emanating from the human body 10 as it travels at the distance D from the human body 10. The sensor array 204 may be switched by a switching mechanism 151 (as shown in FIG. 2) at a point along its travel path to facilitate taking reading from the target body portions.

The electrodes 220 of FIGS. 3A and 4B may each comprise a non-contacting electrode configured to receive signals from an emanating source without requiring electrical or physical contact, such as charge current contact, with the source. In some embodiments, each electrode 220 may comprise an input impedance value sufficient to reliably receive electrical signals at a distance of up to one meter. The electrode 220 may have an input impedance from about 10⁷Ω up to approximately 10¹⁵Ω. The noise floors of high impedance electrodes may be on the order of approximately 4 μV Hz^{-1/2}-70 μV Hz^{-1/2} at 1 Hz, depending on how the electrodes are configured. The use of electrode 220 may allow the detection of body electrical signals in a non-invasive manner.

In the embodiment shown, the electrodes may operate to make a single-ended reading, where there is no charge current contact with the human body and the human body is not grounded. Each electrode may function independently to remotely detect electric potentials created within the human body by electrical activity. In other embodiments, the electrodes may operate in coupled pairs to make readings of the electrical signals based off of differential signals. The noise floor may vary from 4 μV Hz^{-1/2} at 1 Hz when using single ended electrodes to 70 ηV Hz^{-1/2} at 1 Hz when using differential signals from paired electrodes. The embodiments presented here may utilize either single ended or coupled electrodes.

In the embodiment shown in FIG. 3A, the receiving platform 214 may comprise a relatively flat surface for positioning the human body 10 substantially within a distance D from the scanning path 4. The receiving platform 214 may be configured to extend substantially horizontal and parallel to the scanning path 4. It should be recognized by persons of ordinary skill in the art that other configurations of the receiving platform 214 may be utilized such as a standing vertical platform or a bench or seat.

The whole body scanning assembly 200 as shown in FIG. 3A may further comprise an EMF housing 230. The EMF housing 230 may comprise an electromagnetic shield substantially encasing the sensor carrier 202, the scanning mechanism 208, and the receiving platform 214. The EMF housing 230 may shield the sensor carrier 202 and the electrodes 220a-220g (shown in FIG. 4A) from ambient or surrounding electrical signals or other noise that may interfere with accurately reading the electrical signals emanating from the human body 10.

In certain embodiments, the sensor carrier 202 may comprise a connection bundle 215 (shown in FIG. 4B) for incorporating the sensor carrier into the system 150 for characterizing the electrical signals emanating from a human body, as described in FIG. 2. As shown in FIG. 4B, the connection bundle 215 may comprise one or more lead wires which may electrically couple the sensor carrier to the IFC 120 (not shown), which may be located within the EMF housing 230 or may be located remote from the EMF housing 230. In other embodiments, the connection bundle 215 may comprise a wireless receiver and transmitter (not shown) for sending and receiving wireless signals within the system 150.

Turning now to FIG. 5, a hand-held sensor carrier 230 may comprise a handle 234 configured to be grasped by a hand of an operator. The hand-held sensor carrier 230 may be of a size and weight that is suitable for use by the operator. The hand-held sensor carrier 230 may be configured to receive the array 236 of sensors, in a similar fashion as the sensor array 204 of the sensor carrier 208 described in FIGS. 3A and 4A. In the embodiment shown, the array 236 of sensors may comprise an array of twelve sensors 237 arranged in a column-row pattern. Each sensor 237 may comprise a non-contacting electrode as described in FIGS. 3, 4A, and 4B, and the electrodes may be configured either as single ended or coupled into pairs. It should be understood by persons of ordinary skill that the array 236 of sensors may comprise other configurations, such as the single row pattern shown in FIG. 4A

The hand-held sensor carrier 230 may comprise a connection bundle 232 for connecting the sensor carrier to the IFC 120 and carrying the readings corresponding to the electrical signals of the human body 10 received at the array of sensors 204, as shown and described in FIGS. 1 and 2. The connection bundle 232 may include a lead wire for connecting to a receiver, such as IFC 120 as described in FIGS. 1 and 2, or may include a transmitter (not shown) for transmitting wireless signals to the IFC 120.

The hand-held sensor carrier 230 may be incorporated for use as the sensor 152 in the system for characterizing the electrical signals emanating from the human body, as described in FIG. 2. The hand-held sensor carrier 230 may be operated by moving the hand-held sensor carrier 230 along a scanning path that comprises a length of the human body, such as scanning path 4, shown and described in FIG. 3A. In some embodiments, one or more hand-held sensor carriers 230 may be used to characterize the electromagnetic field generated by electrical activity of the human body.

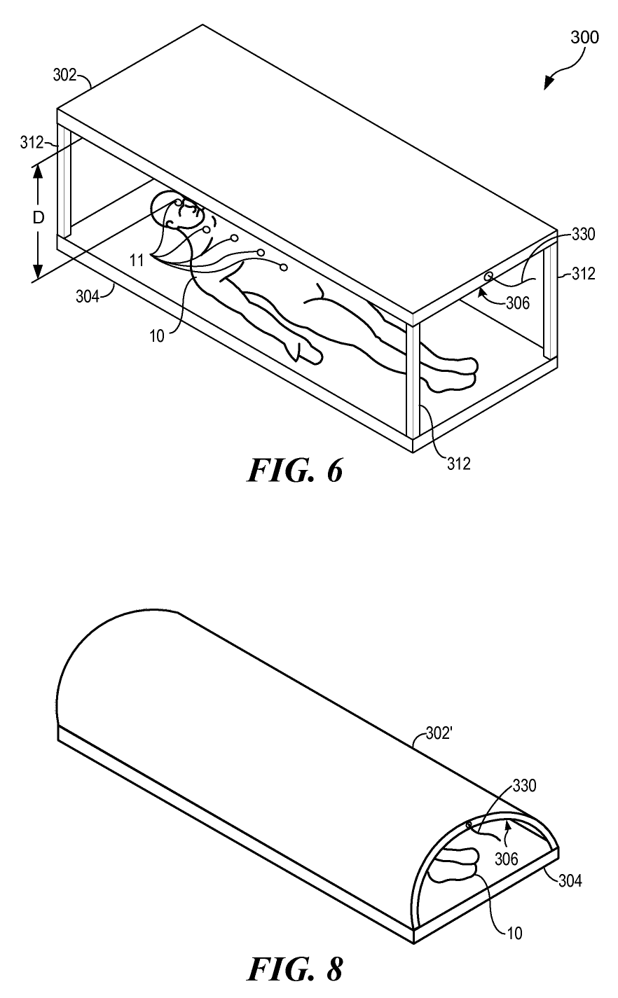

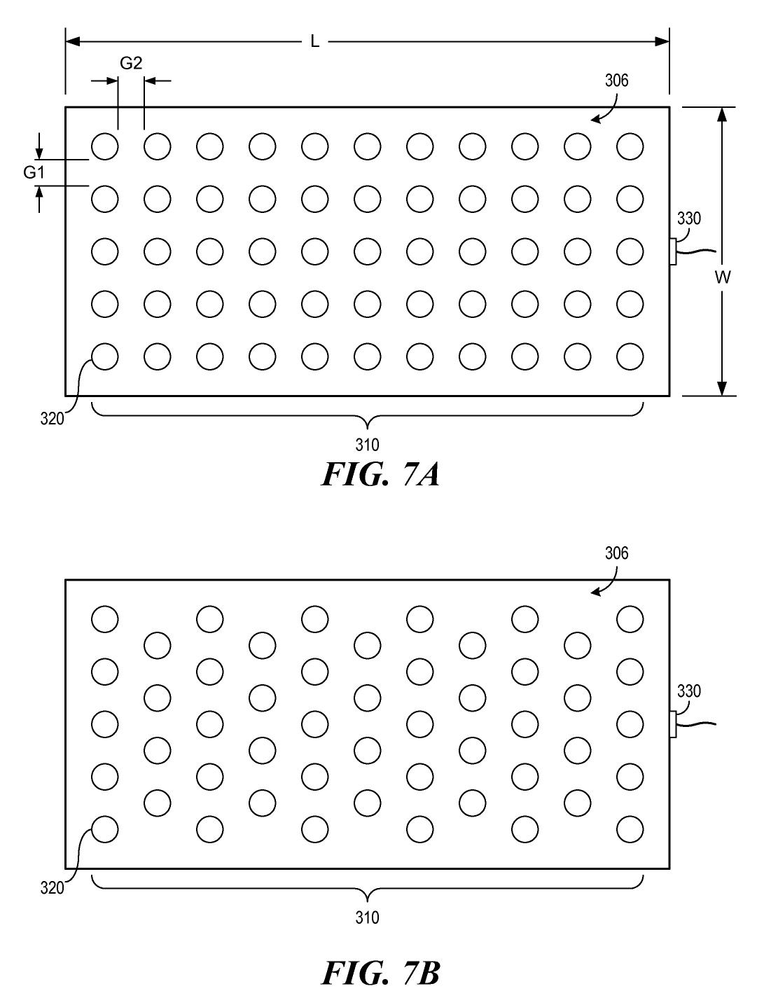

Turning now to FIG. 6, there is shown a perspective view of one embodiment of whole body scanner comprising a stationary electromagnetic (EM) scanner 300. In some embodiments, the stationary EM scanner 300 may take simultaneous or near simultaneous readings of electrical signals emanating from target body portions 11 of a living organism. In the embodiment shown, the stationary EM scanner 300 may comprise a sensor housing 302 that may be positioned at a substantially at a distance D from a living organism, such as human body 10 having target body portions 11, which in some embodiments may correspond to those described in FIG. 1. The sensor housing 302 may comprise a bottom surface 306 configured to receive sensors (shown in FIGS. 7A and 7B) for receiving the electrical signals. The sensor housing 302 may be configured to remain stationary relative to the human 10 as sensors take readings of the EMF of the human body 10.

The human body 10 may be positioned on a support surface 304, which may comprise a substantially flat horizontal surface configured to receive the human body 10. It should be understood that the support surface 304 may comprise other shaped surfaces for supporting the human body 10, while scanning of the electrical signal occurs. Those surfaces may include a seat, a vertical or inclined flat surface, or a molded surface. In still other embodiments, there may be no support surface 304 and the human body 10 may stand at a reference distance from the sensor housing 302, where the sensor housing 302 is positioned to extend vertically such that the bottom surface faces in generally a horizontal direction. It may be advantageous that the human body 10 take a body position such as lying flat or standing straight at generally a distance D from the sensor housing 302 to provide a clear signal from each target body portion.

The sensor housing 302 may comprise a support structure having a plurality of support members 312 for positioning the sensor housing 302 at generally the distance D from the human body 10. In the embodiment shown, the support members 312 may set the sensor housing 302 substantially level relative to the receiving platform 304, so that when the sensor housing 302 may stay at substantially the distance D from the target body portions 11. The support members 212 may be configured to be adjustable to fix the distance D.

Turning now to FIG. 7A, there is shown a first bottom view of a sensor housing 302, such as sensor housing 302 shown in FIG. 6. The bottom surface 306 may be configured for coupling an array 310 of sensors. The sensors of the array 310 may comprise a plurality of electrodes 320 configured for receiving electrical signals emanating from a living organism, such as the human 10, shown in FIG. 6. The electrodes 320 may each comprise a non-contacting electrode, as described above in FIGS. 3A, 4A and 4B, and the electrodes 320 may be configured either as single ended or coupled into pairs.

The electrodes 320 of the array 310 may be arranged in a variety of ways. In some embodiments, a length L of the array 310 may be sufficient to span a height of a human body, from head to toe for instance, and a width W of the array 310 may be sufficient to span width of a human body, such as a shoulder width. The electrodes 320 may be arranged in a column-row fashion, as shown in FIG. 7A. In some embodiments, a first gap G1 may correspond to the distance between each respective row, and a second gap G2 may correspond to the distance between each respective column.

In other embodiments, the electrodes 320 may be arranged in a staggered column-row fashion, as shown in FIG. 7B. It should be understood by persons of ordinary skill in the art that the arrangement of electrodes 320 in the array 310 may be varied to include many patterns, and that the distances between electrodes need not be uniform, but may be grouped or concentrated according to specific target body portions of the human body.

Turning now to FIG. 8, there is shown an embodiment of the sensor housing 302′ having a generally curved shape. The sensor housing 302′ may comprise a partial cylindrical shell which may be mounted to the support surface 304. The array 310 of electrodes 320 (not shown) may be positioned on an inner surface 306 of the sensor housing 302′, in a manner and pattern similar to that shown for sensor housing 302 in FIGS. 7A and 7B. The inner surface 306 and at least a portion of the support surface 304 may define a cavity for receiving at least a portion of the human body 10. In some embodiments, the cavity may receive the entire human body 10 for receiving electrical signals from the human signal source in a manner as described for FIG. 6. It should be understood by persons of ordinary skill in the art that the shape of the sensor housing 302′ may take other configurations, such as a general dome shape, or a contoured shape that generally conforms to the shape of the human body 10. In some embodiments, the shape of the sensor housing 302′ may facilitate providing a generally uniform distance between the electrodes 320 and the human body 10.

The support surface 304, as shown in FIGS. 6 and 8, may be configured to move the human body 10 relative to the sensor housings 302 and 302′. The sensor housings 302 and 302′ may remain stationary while the human is moved relative to the array 310 of electrodes 320. One advantage of this configuration may be that the number of electrodes 320 may be decreased.

The sensor housings 302 and 302′ may be located within a shielding, such as the EMF housing 230 shown in FIG. 3A. The shielding (not shown) may reduce electromagnetic noise picked up by the electrodes 320, shown in FIGS. 6, 7, and 8. In other embodiments, the electrodes 320 may comprise a low noise floor (approximately 4 ηV Hz^{-1/2} to 70 ηV Hz^{-1/2} at 1 Hz), which may eliminate the need for separate noise shielding.

In certain embodiments, the sensor housings 302 and 302′ may comprise a connection bundle 330 (shown in FIGS. 6, 7, and 8) for incorporating the sensor housings 302 and 302′ into the system 150 for characterizing the electrical signals emanating from a human body, as described in FIG. 2. As shown in FIGS. 6, 7, and 8, the connection bundle 330 may comprise one or more lead wires which may electrically couple the sensor housings 302 and 302′ to the IFC 120, which may be located within an EMF housing (not shown) or may be located remote from the EMF housing. In other embodiments, the connection bundle 330 may comprise a wireless receiver and transmitter (not shown) for sending and receiving wireless signals within the system 150.

The sensor housings 302 and 302′ (shown in FIGS. 6, 7, and 8) may be configured to be turned on by a switching mechanism 151 (as shown in FIG. 2). A human body 10 may be positioned on the support surface 304 (as shown in FIG. 6) so that the target body portions are in a position relative the array 310 to facilitate taking readings of the electrical signals.

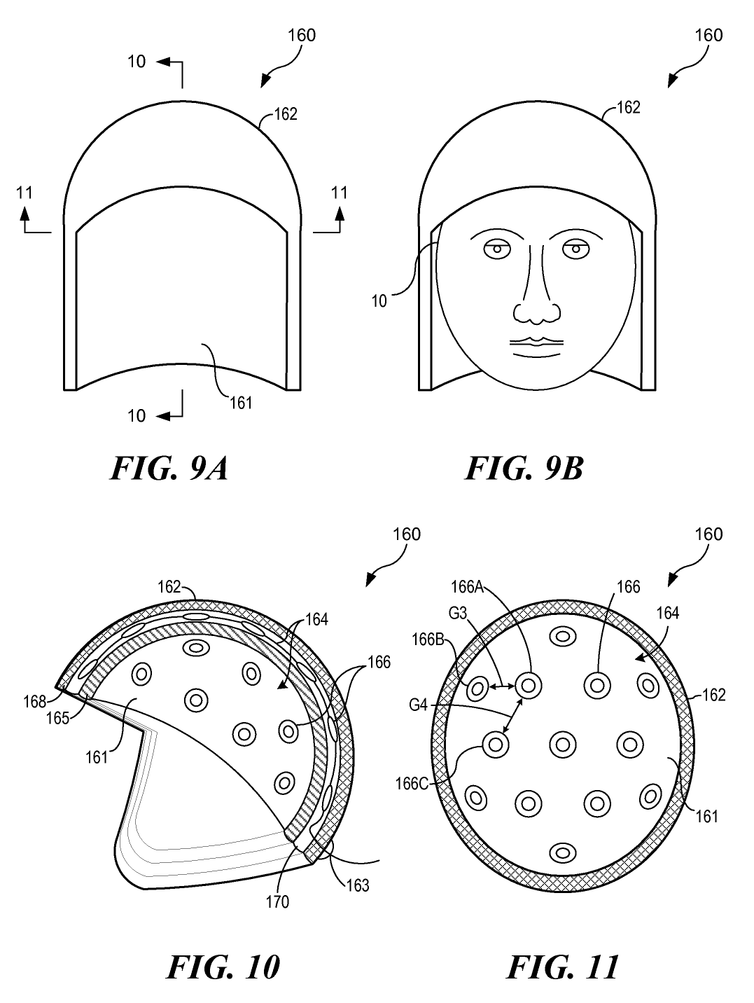

Turning now to FIG. 9A, there is shown a perspective view of another embodiment of a whole body scanner assembly 160. The whole body scanner assembly 160 may comprise a covering, or garment configured to position an array of electrodes substantially adjacent to the human body 10. In the embodiment shown in FIG. 9A, the whole body scanner assembly 160 may comprise a covering shaped like a helmet 162 configured to be worn on a head portion of the human body 10. The helmet 162 may be shaped to cover target areas of the human body 10, where electromagnetic activity is expected. The helmet 162 may comprise a shell having an inner surface 161 forming a cavity for receiving and covering the head portion. In the embodiment shown in FIG. 9B, the helmet 162 covers a cranial area of the head portion of the human body 10. The cranial area may comprise target areas such as the crown and forehead of the head portion of the human body 10. These areas it is expected will provide concentrated sources of electrical signals indicating the electrical activity of the human body 10.

Turning now to FIGS. 10 and 11, there are shown cross-sectional views of the helmet 162 taken along lines 11-11 and 12-12, as shown in FIG. 9A. The helmet 162 may comprise an array 164 of sensors 166 on the inner surface 161 of the helmet 162. The array 164 may be positioned on the inner surface 161 of the helmet. As shown in FIG. 11, the array 164 may comprise a pattern, such as a evenly scattered pattern that follows inner contours of the inner surface 161. In the embodiment shown, each sensor 166a may have one or more pre-defined distances, such as G2 and G3, from one or more adjacent sensors 166b and 166c. In other embodiments, the pattern may be varied to concentrate the sensors 166 around target areas of the head portion of the human body 10.

The sensors 166 shown in FIGS. 10 and 11 may comprise non-contacting electrodes as described in FIGS. 1 and 2. These sensors 166 may not require charge contact with a skin surface of the head portion.

As shown in FIG. 10, the helmet 162 may comprise a plurality of layers of material. In some embodiments, an outer layer 168 may include material for shielding the sensors 166 from electromagnetic noise. The outer layer 168 may be constructed from fabric lined with mu metal or other alloys, or other suitable material known by persons of ordinary skill in the art as having electromagnetic shielding qualities.

A middle layer 170 may comprise a fabric including the array 164 of sensors 166. The sensors 166 may be mechanically coupled together with tethers (not shown) or braces to assist in maintaining their relative spacing. The array 164 may further comprise a connection bundle 163 which may comprise one or more wires configured to receive and transmit electrical signals from and to the sensors 166. The connection bundle may allow the whole body scanner 160 to be incorporated into a system for characterizing the electromagnetic field emanating from a human body, such as the system described in FIGS. 1 and 2.

The sensors 166 may remain electrically isolated from each other and from the electrical currents of the human body 10. In FIG. 10, there is shown a cross-section view of the helmet 162 showing the inner surface 161 with the position of the sensors 166 represented by concentric circles. These concentric circles are shown merely for illustrative purposes and may not represent that the electrodes pass through the inner surface 161. In some embodiments, the sensors may be embedded in the middle layer 170 between an inner layer 165 and the outer layer 168.

The inner layer 165 of the helmet 162 may comprise an insulating layer for preventing charge contact with the human body. The inner layer may comprise various materials, such as cotton or wool or other suitable material for distancing the electrodes from the charge currents of the human body 10. It may be advantageous to use a material for the inner layer that allows electromagnetic signals to pass, but does not allow charge currents. Materials that are used in the outer layer 168 may not be appropriate for use in the inner layer 165, since the outer layer 168 may be used as a shield from electromagnetic noise, while the inner layer may be used to facilitate the readings that the sensors 166 make.

In some embodiments, the inner layer may include padding to distance the sensors 166 from the electrical currents of the human body 10. A gap between the sensors 166 and the skin surface may also provide an insulation from the electrical currents of the human body 10.

The sensors 166 positioned in the middle layer 170 may be coupled to the either the material of the outer layer 168 or the material of the inner layer 165. In some embodiments, the array 164 may be coupled to the outer layer to anchor the position of the array 164 of sensors 166 to the structure of the helmet. The sensors 166 may be held in the same position relative to the target areas of the human body 10 by being rigidly coupled to the inner surface 161 of the helmet 162. The frictional and static contact of the inner surface 161 may also hold the array 164 of sensors 166 in place relative to the human body 10.

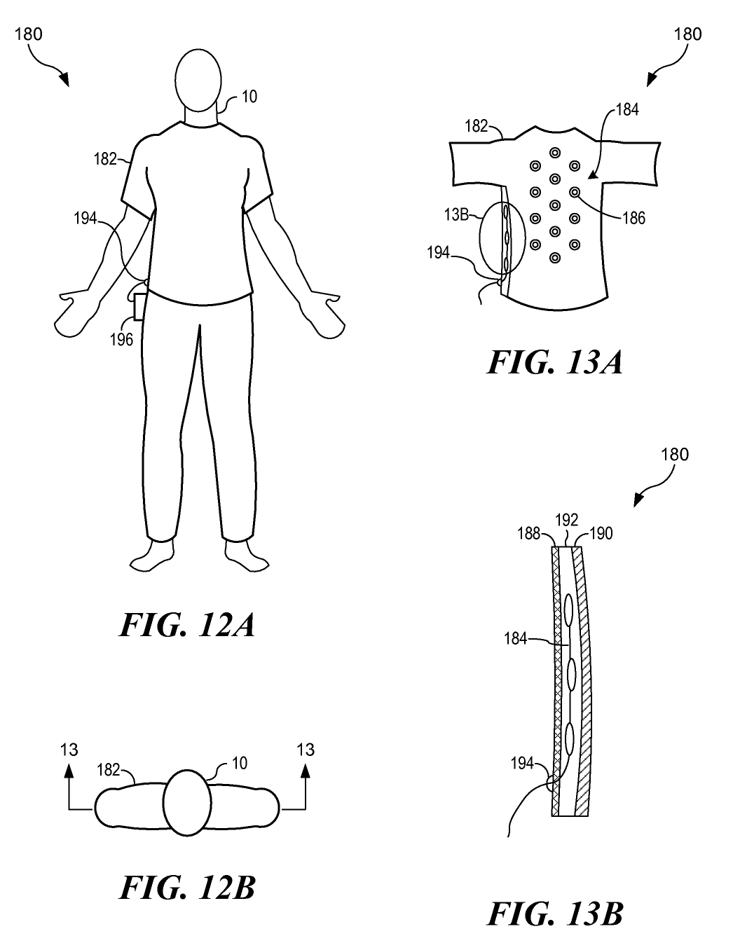

Turning now to FIG. 12A, there is shown another embodiment of a whole body scanner 180 comprising a garment, such as a shirt 182. The shirt 182 may be configured to position an array of sensors substantially near a torso portion of a human body 10. The array of sensors may reside within the fabric of the shirt 182 or between layers of fabric. It is intended that the shirt 182 may fit a human subject and be worn while readings corresponding to the electrical signals emanating from the human subject are made. FIG. 12B shows a top view of the shirt 182, as worn by a human body 10.

Turning now to FIG. 13A, there is shown a cross-sectional view of the shirt 182 taken along line 13-13, as shown in FIG. 12B. The shirt 182 may be configured with an array 184 of sensors 186 on an inner surface 188 of the shirt 182. The sensors 186 may be configured to receive electrical signals emanating from the human body 10.

The sensors 186 shown in FIG. 12 may comprise non-contacting electrodes as described in FIGS. 1 and 2. These sensors 186 may not require charge contact with a skin surface of the human body 10.

Turning to FIG. 13B, there is shown a zoomed view of Section 13B shown in FIG. 13A. As shown in FIG. 13B, the array 184 may be positioned between an outer layer 188 and an inner layer 190 in a middle layer 192 in a manner of the layers 165, 170 and 168 described for the helmet 162. A connection bundle 194 may comprise one or more wires for incorporating the whole body scanner 180 into a system for characterizing the electromagnetic field emanating from a human body as described in FIGS. 1 and 2.

It should be understood by persons of ordinary skill, that other garments may be configured to integrate an array of sensors, such as those described in FIGS. 9A, 9B, 10, 11, 12, 13A, and 13B. Such garments configured to operate as a whole body scanner may include pants, wrist bands, or head bands, socks, shoes, or other garment intended be worn on the human body. In still other embodiments, the garment may be configured to communicate with a processor, such as the interface 120, shown in FIG. 2, by wireless signals.

Turning back to FIG. 12A, the connection bundle 194 may be coupled to a processor 196 configured to be worn or attached to the human body 10 or a separate garment worn by the human body 10. The processor 196 may operate with the same or similar function of the interface 120 and first computer 124, as described in FIGS. 1 and 2. In other embodiments, the processor 196 may be comprised of one or more processors or circuits configured to transmit wireless signals to a remote computer network for processing, filtering, amplifying, storing or displaying a characterization of the electrical activity of the human body.

In other embodiments, one or more garments configured in similar manner as the helmet 162 and the shirt 182 may be networked to operate as a single unit so that a complete characterization of the electrical activity of the human body may be made.

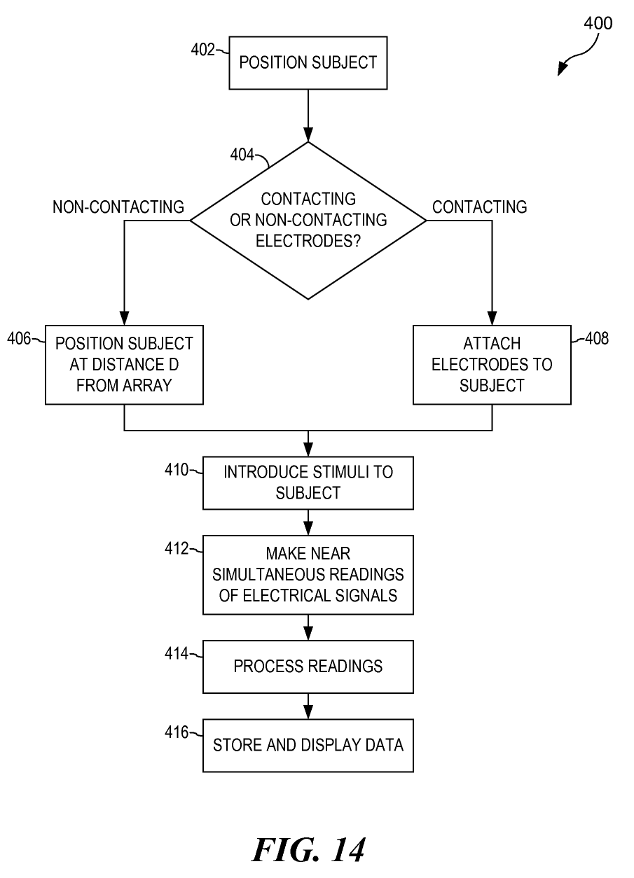

Turning now to FIG. 14, there is shown a flow diagram of a method 400 for characterizing the electric signal emanating from a living organism. An operator may assist in this method, or it may be automated such that a human subject alone may perform the method using an apparatus for characterizing the electrical signals emanating from a living organism, such as those described in FIGS. 1-13B. The characterization may be used to assist in assessing certain medical, psychological, or other physiological conditions or responses to certain stimuli.

In operation 402, the human subject may be positioned on a support surface, such as support surfaces 214 and 304 shown in FIGS. 3 and 6. In operation 404, either non-contacting electrodes are utilized, such as electrodes 320, shown in FIGS. 3 and 6, or contacting electrodes, such as electrodes 104, shown in FIG. 1. When using non-contacting electrodes, such as in operation 406, the human subject may be positioned substantially within a distance D from an array of electrodes. In some embodiments, such positioning may involve mechanically adjusting the position of the array of electrodes or mechanically adjusting the position of the support surface, or it may involve moving the human subject relative to the array. In the case where a hand held sensor carrier is used, such as that shown in FIG. 5, or where the subject is standing apart from a fixed array of electrodes, the human subject may move or be positioned to an optimum distance D by the operator.

In some embodiments, the distance D may be varied to capture readings of the electrical signal which may vary with the distance D of the electrodes from the human body. For instance, when the electrodes are placed closer to the skin surface of the human subject, the readings may be of the electrical voltages directly from the body surface. As the distance D is increased, the readings of electrical signals may be from other electric fields generated by the human body.

In some embodiments, the distance D may be chosen to provide clearance to all portions of the body. For instance, where a moving sensor carrier is used, such as that described in FIGS. 3A, 4A and 4B, the distance D may be at least one foot from the human subject. This distance D may provide for clearance from the extremities of most human subjects and still provide for accurate readings.

When using contacting electrodes, such as in operation 408, the electrodes may be placed in physical or charge contact with the human subject. The human subject may be prepped to receive the electrodes, according to standard methods of connecting the electrodes to a human body. The electrodes may be attached to the target body portions, such as in the manner described in FIG. 1.

The operator may further use test or calibration data taken from the electrodes to ensure that the readings from the electrodes will be accurate. Such calibration may include taking a reference reading of the electrical signal prior to introducing a stimulus to the subject. It should be understood that the reference signal need not be taken contemporaneously with the positioning of the human subject. In some cases, it may have been taken at a prior visit of the human subject.

In the method 400, one or more stimulus may be introduced to the human subject at operation 410. Such stimulus may include examination of the electromagnetic field in the context of a pre-existing disease or other health condition, such as depression, so that variations in the electromagnetic field emanating from the human subject may be monitored over a course of time. Stimulus may also include certain medical, psychological, or other treatment so that the response as characterized by the electrical field is monitored over a course of time. Other stimulus may be environmental, such as sounds, visual cues, verbal cues or questions posed to the subject, smells, or touch sensations.

In operation 412, the electrodes, whether contacting or non-contacting, may make contemporaneous, near simultaneous or simultaneous readings of the electromagnetic field of the subject. The readings may span a discrete time period or may be taken in increments of time, such as one second. In the case of the embodiment shown in FIG. 1, the contacting electrodes may be configured to be switched to simultaneously receive electrical signals. In some embodiments, a switching mechanism may receive a command to put the electrodes in an active reading mode or put the electrodes in a standby mode.

In the case of the embodiment shown in FIGS. 3A, 3B, 3C, and 3D, the scanning mechanism 208 may move the sensor carrier 202 along the scanning path 4 to take readings of the electrical signal along the entire length of the human subject. The speed at which the sensor carrier 202 is moved may be varied, and in some embodiments, increasing the speed of the sensor carrier 202 may allow the readings taken from one end of the scanning path 4 to the other end to be contemporaneous to nearly simultaneous by decreasing the time taken for the sensor carrier 202 to span the scanning path 4. Similarly, using the hand held sensor carrier 230 shown in FIG. 5, the operator may manually sweep a scanning path across a length of the human subject.

In the case of the embodiment shown in FIG. 6, the sensor housing 302 may remain stationary. The non-contacting electrodes shown may be simultaneously switched to a read configuration by a switching mechanism. Each of the electrodes may receive electrical signals from the human subject. In some embodiments, the human subject may be moved relative to the stationary sensor housing.

It should be understood that in some environments, an electromagnetic housing, such as EMF housing 230 shown in FIG. 3A may be used as an electromagnetic shield during the scanning process and for any of the embodiments herein described. The housing may provide shielding from noise in the environment and may help make accurate readings of the electrical signals emanating from the human subject. It may be an additional advantage in using the non-contacting electrodes herein described, because an electromagnetic shield may not be required, but may be merely optional, or may require less shielding than the conventional contacting electrodes.

In operation 414, the electrodes, whether contacting or non-contacting, may receive and transmit data corresponding to the electrical signals of the human subject. This data may be received by a processor, such as the IFC 120 as described in FIGS. 1 and 2. The processor may perform functions such as interpreting the data corresponding to the received electrical signals. The processor may provide data relating to waveforms of electrical potential of the body as characterized in a time domain. The processor may further interrelate or couple the electrical signals from multiple portions of the human subject occurring in real-time so that a complete picture of the human body signal under given conditions may be recorded.

In operation 416, the processor may store data in a memory, such as a hard drive or other memory device. Such storage may include uploading via private computer network or the internet to a remote storage location, using either wired or wireless technology. In other embodiments, the processor may display data in a user readable format such at that described in FIG. 2.

The method 400 described in FIG. 14, may be modified for a wearable embodiment of the whole body scanner 160 and 180, as described in FIGS. 9-13B. In some embodiments, the wearable whole body scanner 160 and 180 may comprise one or more garments configured with an array of sensors. A human subject may put the one or more garments onto a body portion intended for that garment, such placing the helmet 162 onto the head portion of the human body. A switch, such as switch mechanism 151, shown in FIG. 2, may be used by the human subject or remotely switched by an operator. The arrays of sensors of the one or more garments may be configured to make readings corresponding to the electrical signals emanating from the human body. The readings may be stored in a processor worn on the human subject or transmitted to a computer network. These readings may be stored, processed or displayed in a manner similar to that described in operations 414 and 416 of FIG. 14.

There may be certain advantages to scanning target body portions of the human body according to the method 400. For instance, taking contemporaneous, near simultaneous or simultaneous readings may allow researchers to study electromagnetic signal traffic between target body portions. The signal traffic between body portions may be characterized in a variety of contexts. A stimulus, such as a visual cue, may be introduced to a human test subject to characterize the electromagnetic response of target body portions, such as the celiac ganglion and hypogastric (sacral) plexus. It should be understood by persons of ordinary skill in the art that signal traffic may be characterized according to different combinations of target body portions and under different conditions.

Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure. In some instances, some features of the present invention may be employed without a corresponding use of the other features. Many such variations and modifications may be considered desirable by those skilled in the art based upon a review of the foregoing description of preferred embodiments. Accordingly, it is appropriate that the appended claims be construed broadly and in a manner consistent with the scope of the invention.

I claim:

1. A system for characterizing electrical signals from a human signal source, wherein the human signal source comprises a human body transmitting electrical signals as result of the electrical activity of the human body, the system comprising:
an array of sensors configured to be positioned to receive the electrical signals from the human signal source, wherein the array is configured to transmit readings corresponding to the electrical signals; and
a processor coupled to the array of sensors for interpreting the readings of the electrical signals from the human signal source, wherein the processor is further configured to output a characterization of the electrical signals.

2. The system of claim 1, wherein the array of sensors comprises a first sensor configured to be attached to a head portion of the human body, a second sensor configured to be attached to a neck portion of the human body, a third sensor configured to be attached to a solar plexus region of the human body, and a fourth sensor configured to be attached to a sacral plexus of the human body.

3. The system of claim 1, wherein the array of sensors comprises one or more sensors positioned in a row configured to be substantially transverse to the length of the human body and to be positioned substantially equidistant from the human body, such that there is a gap between the array and the human body.

4. The system of claim 3, wherein the array of sensors is configured to scan between the head portion of the human body and the foot portion of the human body in order to receive contemporaneous electromagnetic signals from different portions of the human body.

5. The system of claim 1, wherein the array of sensors is configured as a plurality of sensors arranged in a row and column pattern to cover a surface area of the human body and positioned substantially equidistant from the human body to receive simultaneous electromagnetic signals from different portions of the human body.

6. The system of claim 3, wherein each sensor of the array of sensors comprises an electrode.

7. The system of claim 4, wherein each sensor of the array of sensors comprises a sensor configured to detect the electrical signals without making electrical contact to the human signal source.

8. The system of claim 7, wherein the sensor is further configured to have an impedance value in the range of approximately 10⁷Ω to 10¹⁵Ω.

9. The system of claim 5, wherein each sensor of the array of sensors comprises a sensor configured to detect the electrical signals without making electrical contact to the human signal source.

10. The system of claim 9, wherein the sensor is further configured to have an impedance value in the range of approximately 10⁷Ω to 10¹⁵Ω.

11. A method for characterizing signals from a human signal source, the method comprising:
receiving from an array of sensors a plurality of readings corresponding to electrical signals emanating from a human signal source;
processing the plurality of readings in a processor electrically coupled to the array of sensors; and
outputting from the processor a characterization of the electrical signals.

12. The method of claim 11 further comprising:
attaching a first sensor of the array of sensors to a head portion of a human body, a second sensor of the array configured to be attached to a neck portion of a human body, a third sensor of the array configured to be attached to a solar plexus region of a human body, and a fourth sensor of the array configured to be attached to a sacral plexus of a human body.

13. The method of claim 11 further comprising:
positioning the array of sensors substantially along a line transverse to a length of a human body and at a distance from the human body that defines a substantially equidistant gap from the array of sensors to the human body; and
moving the array of sensors along the length of the human body, wherein the plurality of readings are received by the array of sensors while the array of sensors is moved, and wherein the plurality of readings comprise readings corresponding to contemporaneous electrical signals emanating from the human body.

14. The method of claim 11 further comprising:
positioning the array of sensors to cover an area of the human body at a distance from the human body that defines a substantially equidistant gap from the array of sensors to the human body.

15. The method of claim 14, wherein receiving from an array of sensors a plurality of readings corresponding to electrical signals emanating from a human signal source further comprises maintaining the equidistant gap during the receiving of electrical signals without any relative movement between each sensor of the array and the human body, and wherein the plurality of reading comprise readings corresponding to near simultaneous electrical signals emanating from the human body.

16. The method of claim 15 further comprising:
arranging the array of sensors in a column-row arrangement to cover the area of the human body.

17. The method of claim 16, wherein each sensor of the array of sensors is operationally fixed to a carrier member.

18. The method of claim 11, wherein each sensor comprises an electrode configured to receive a reading corresponding to the electrical signals emanating from the human body.

19. The method of claim 13, wherein each sensor of the array of sensors is a non-contacting sensor having an impedance value that allows the non-contacting sensor to detect the electrical signals without making electrical contact to the human signal source.

20. The method of claim 16, wherein each sensor of the array of sensors is a non-contacting sensor having an impedance value that allows the non-contacting sensor to detect the electrical signals without making electrical contact to the human signal source.
